(19) United States
(12) Patent Application Publication
(10) Pub. No.: US 2006/0115495 A1
(43) Pub. Date: Jun. 1, 2006

(54) Protein-noble metal nanoparticles

(75) Inventors: Miguel Jose Yacaman, Lakeway, TX (US); Jose Luis Elechiguerra, Austin, TX (US); Humberto Herman Lara, San Nicolas de los Garza (MX); Justin Lockheart Burt, Austin, TX (US)

Correspondence Address: CHALKER FLORES, LLP, 2711 LBJ FRWY, Suite 1036, DALLAS, TX, 75234, US

(73) Assignee: Board Of Regents, The University of Texas System, Austin, TX (US)

(21) Appl. No.: 11/271,392

(22) Filed: Nov. 11, 2005

Related U.S. Application Data
(60) Provisional application No. 60/627,372, filed on Nov. 12, 2004.
(60) Provisional application No. 60/708,732, filed on Aug. 16, 2005.

(51) Int. Cl. A61K 39/12

(52) U.S. Cl. 424/204.1; 424/186.1

(57) ABSTRACT

The present invention is a composition and method of making protein-noble metal nanoparticles and methods for using the same as anti-virals.

This application claims priority to U.S. Provisional Patent Application Ser. No. 60/627,372, filed Nov. 12, 2004 and U.S. Provisional Patent Application Ser. No. 60/708,732, filed Aug. 16, 2005, the entire contents of each are incorporated herein by reference. Without limiting the scope of the invention, its background is described in connection with nanoparticles.

### TECHNICAL FIELD OF THE INVENTION

The present invention relates in general to the field of antivirals, and more particularly, to compositions, methods and treatment of viral particles with silver nanoparticles to reduce or eliminate viral infection and/or transmission.

### BACKGROUND OF THE INVENTION

In 2003, an estimated 4.8 million people (range: 4.2-6.3 million) became newly infected with HIV. This is more than in any one year before. Today, some 37.8 million people (range: 34.6-42.3 million) are living with HIV, which killed 2.9 million (range: 2.6-3.3 million) in 2003, and over 20 million since the first cases of AIDS were identified in 1981.

Women increasingly infected by HIV. In recent years, the overall proportion of HIV-positive women has steadily increased. In 1997, women were 41% of people living with HIV; by 2002, this figure rose to almost 50%. This trend is most marked in places where heterosexual sex is the dominant mode of transmission, particularly the Caribbean and sub-Saharan Africa. Women also significantly figure in many countries with epidemics that are concentrated in key populations such as injecting drug users, mobile populations, and prisoners.

AIDS is a fatal catastrophic disease that presently infects millions of people worldwide. Although initially concentrated in central Africa and in certain high risk groups in other geographic areas including the United States, AIDS is now spreading to other areas and is appearing in individuals who are not members of the recognized risk groups. As a result, major efforts are being made to develop methods of preventing the transmission of AIDS, methods of curing AIDS once contracted, and methods of ameliorating the symptoms of AIDS. To date, however, AIDS has been proven difficult to treat or prevent.

AIDS is caused by a virus. This virus has been referred to by a number of names in the literature, including HIV (human immunodeficiency virus) LAV (lymphadenopathy-associated virus), ARV (AIDS-related virus) and HTLV-III (human T-cell leukemia virus-III).

It is generally known that viruses can be divided into two groups based upon the nature of the virus' genetic material. Some viruses are DNA viruses, that is there genetic material is deoxyribonucleic acid, while others are RNA (ribonucleic acid) viruses. The RNA viruses can further be divided into two groups, those in which replication of the viral genome proceeds by making an RNA copy directly from the RNA genome and those in which a DNA intermediate is involved. This latter type of RNA virus is called a retrovirus. The AIDS virus is a retrovirus. Thus, like other retroviruses, it has an enzyme called reverse transcriptase (or RNA-dependent DNA polymerase) which catalyzes transcription of viral RNA into double helical DNA. This DNA sequence is integrated into the genome of the infected cell where it is known as a provirus. Subsequent transcription of this provirus by the transcription mechanism of the infected cell produces new viral RNA for packaging into new virus particles.

Because the AIDS virus may lie dormant in an infected cell in the form of a provirus for extended periods of time, it has been difficult to establish the precise routes by which AIDS is spread. It is known, however, that AIDS can be transmitted to a person by transfusing that person with blood containing the AIDS virus. AIDS can also be transmitted to a person through homosexual or heterosexual intercourse with a partner infected with the AIDS virus. Transmission of the AIDS virus is facilitated by preexisting sexually transmitted diseases (STD's) other than AIDS, for example gonorrhea. Further, scientists suspect that the AIDS virus is spread easily during sexual intercourse due to tearing of tissue which would abet entry of the AIDS virus into the blood stream.

### SUMMARY OF THE INVENTION

In response to the growing threat of AIDS transmission, the use of condoms during sexual intercourse has been suggested as a way of preventing transmission of the AIDS virus. Improper use of condoms, or their perforation during intercourse, renders them only partially effective. Accordingly, there is a pressing need for a better method of inhibiting the transmission of the AIDS virus in humans during sexual intercourse and during surgical procedures on infected patients. The present invention provides compositions and methods for making and using an anti-viral composition for use in treating and preventing viral infection.

The present inventors demonstrate herein the properties of unique silver nanoparticles, in different forms, to deactivate HIV with concentrations below the cytotoxic concentrations of, e.g., MT2 cells. Therefore, the present invention provides for an inexpensive, easily available composition and convenient method of inhibiting the transmission of the AIDS virus in humans as a result of sexual intercourse. The method of the present invention relies upon the action of antiviral compositions comprising silver nanoparticles. Silver nanoparticles are effective to reduce the infectivity of the AIDS virus and also kill the causative organisms of many other sexually transmitted diseases (STD). The invention is therefore useful to reduce the immediate risk of AIDS transmission.

AIDS is caused by a virus. This virus has been referred to by a number of names in the literature, including HIV (human immunodeficiency virus), LAV (lymphadenopathy-associated virus), ARV (AIDS-related virus) and HTLV-III (human T-cell leukemia virus-III). For simplicity, the virus causing AIDS will be referred to herein as the AIDS virus.

The method of the present invention relies upon the action of silver nanoparticles, which results in a rapid killing action within minutes. The invention is therefore useful to reduce the immediate risk of AIDS transmission. It also reduces future risk of AIDS transmission by eliminating STD causing organisms which increase the risk of AIDS.

The present inventors have also developed a method for the production of a novel class of nanomaterials, namely, protein-conjugated noble metal nanoparticles. Examples of uses for these nanoparticles include, e.g., noble-metal nanoparticles directly conjugated to proteins, e.g., globular protein molecules that may be used for targeting of, e.g., cancerous cells, and for use of this nanomaterial for antiviral and antibacterial applications. For specific targeting the present invention may further include one or more targeting molecules (e.g., cytokines) that bind specifically to target cells, e.g., those cells that express the cognate receptor for the cytokine.

First, the inventors developed a method for synthesizing water-soluble noble metal nanoparticles, grown within and directly functionalized by bovine serum albumin (BSA), a globular protein, without the use of additional protecting or linking agents. Both gold and silver nanoparticles have been produced using this method, and the synthesis method can easily be extended to other noble metals, such as the platinum group metals. Other globular proteins such as human serum albumin (HSA) can also be and/or substituted for BSA, e.g., immunoglobulins, cytokines, receptors, lectins, glycoproteins, lipoproteins, toxins, toxoids, collagen, proteins with RGD sequences, bacterial proteins, viral proteins, parasitic proteins, and fusion proteins and portions thereof. The method of synthesis includes a chemical reduction of an ionic metal precursor at room temperature in aqueous solution, in the presence of, e.g., a globular protein, e.g., bovine serum albumin (BSA). Under proper pH conditions, disulfide bonds of the protein are available for direct bonding with the noble metal nanoparticles. It was found that the polypeptide backbone of the protein was intact, and the method did affect the functional groups of the constituent amino acid residues. In one example, sodium borohydride was used as a reducing agent, however, other more gentle reducing agents such as ascorbic acid and glucose may be used with the present invention.

Certain advantages of the present invention over known bioconjugate preparation techniques were found. The method disclosed herein permits a nearly quantitative yield of well-dispersed nanoparticles directly conjugated to globular protein molecules. Furthermore, the resulting nanoparticles exhibited robust dimensional stability and ease of handling and storage as a powder, rare properties for bioconjugated nanomaterials. Also, it was found that the nanoparticles formed by the method are stabilized by a combination of sulfur bonding with the protein and steric protection from the globular protein. Therefore, the particles have free surface area for interaction with external species.

Next, it was found that the protein-conjugated nanoparticles were stable in aqueous solution at room temperature. For example, for use in long-term storage, the water content may be evaporated, spray-dried, freeze-dried, vacuum dried, heat-vacuum dried, etc., and the product ground into a fine powder for storage purposes without inducing nanoparticle coalescence. The nanoparticle size distribution is narrow and well-controlled, with average diameters ranging from 1-2 nm in the case of gold, and 2-5 nm for silver nanoparticles, depending upon the reaction conditions. The present invention may also take advantage of one or more biodegradable and/or biocompatible polymers for use in chemical, catalytic, therapeutic, pharmaceutic or other application for extended release, enhanced stability, longer half-life and the like. Noble metal nanoparticles in this size range are extremely desirable because of their unique physicochemical properties including: a large surface to volume ratio, which provides a more efficient use of material that will be available for reaction with external species; they exhibit tunable changes in electronic structure due to quantum size effects; and the structure was constantly fluctuating, which increased their catalytic activity.

Another advantage of the present invention is that serum albumin is a globular protein, and is the main protein component found in blood plasma, therefore, serum albumin is readily available and very unlikely to be immunogenic. In the body, serum albumin is confined to the circulatory system. When nanoparticles are directly conjugated to serum albumin molecules, may be used to target delivery within the bloodstream. The albumin-metal nanoparticles disclosed herein may also be conjugated to antibodies, e.g., humanized antibodies, using well-known procedures. Functionalized protein-nanoparticle conjugates with antibodies allow for enhanced targeting. The serum albumin-conjugated noble metal nanoparticles are well-suited for easy incorporation into gels and creams for a variety of applications.

### BRIEF DESCRIPTION OF THE DRAWINGS

For a more complete understanding of the features and advantages of the present invention, reference is now made to the detailed description of the invention along with the accompanying figures and in which:

FIGS. 1a through 1e show the toxicity of silver nanoparticles in MT-2 cells. FIGS. 1a and 1b are graphs that those the toxicity of the nanoparticle preparations against MT-2 cells after 3 hours (1a), and 24 hours (1b), as determined using the Trypan Blue exclusion assay. FIGS. 1c-1e, Optical microscopy images of MT-2 cells after 24 h of exposure to carbon coated silver nanoparticles at silver nanoparticle concentrations of 3 μg/mL (1c), 12 μg/mL (1d) and 50 μg/mL (1e) (10× magnification);

FIGS. 2a through 2h shows the inhibition of HIV-1 by silver nanoparticles. FIGS. 2a and 2b are graphs that show the toxicity of the nanoparticle preparations with CD4+ MT-2 cells (2a), and cMAGI cells (2b) of the three preparations (prior art carbon-coated particles) and the two particles of the present invention. The micrographs are as follows: 2c, MT-2 cells negative control; 2d, MT-2 cells positive control; 2e, HIV-1 treated MT-2 cells where the virus was previously exposed to carbon coated silver nanoparticles at a concentration of 6 μg/mL; 2f, cMAGI cells negative control; 2g, cMAGI cells positive control and h, HIV-1 treated cMAGI cells where the virus was previously exposed to carbon silver nanoparticles at a concentration of 6 μg/mL (10× magnification);

FIGS. 3a-3l are HAADF images of the HIV-1 virus, namely, FIGS. 3a-3c, HIV-1 virions without nanoparticle treatment; 3d-3f, HIV-1 virions exposed to carbon-coated silver nanoparticles; and 3g-31, HIV-1 virions exposed to BSA-conjugated silver nanoparticles;

FIGS. 4a through 4f summarize the structure-function relationship of the interaction between the coated silver nanoparticles of the present invention and HIV virions. FIG. 4a is an HAADF image of an icosahedral virus with regular spatial relationships clearly observed among attached nanoparticles (scale bar: 20 nm); 4b is a structural model of an icosahedral HIV-1 virion (circles represent the positions of glycoprotein gp120); 4c, shows the tertiary structure of gp120 determined by Kwong, et al. (PDB 1GC1); 4d is another shematic representation of a silver nanoparticle binding with the gp120 subunit of the HIV-1 envelope glycoprotein; 4e is a graph that summarizes the size distribution of silver nanoparticles bound to the HIV-1 virus, derived from all tested preparations; and 4f is an EDS analysis of image a confirming the presence of Ag (the C signal comes from both the TEM grid and the virus, O, and P are from the virus, and Na, Cl, and K are present in the culture medium. Ni and Si come from the TEM grid, while Cu is attributed to the sample holder;

FIG. 5 is a graph that shows the change in intensity of the protein-coated silver nanoparticles of the present invention made with different molar ratios of, e.g., BSA and the silver nanoparticles of the present invention;

FIGS. 6a through 6c are as follows: 6a is a transmission electron micrograph; 6b High Angle Annular Dark Field image; and 6c a particle size distribution histogram of carbon-coated silver nanoparticles of the prior art;

FIGS. 7a through 7c are as follows: 7a is a transmission electron micrograph; 7b is a High Angle Annular Dark Field image; and 7c is a particle size distribution histogram, of PVP-coated silver nanoparticles developed by the present inventors; and

FIGS. 8a through 8c are as follows: 8a is a transmission electron micrograph; 8b is a High Angle Annular Dark Field image; and 8c is a particle size distribution histogram of the Albumin-coated silver nanoparticles of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “antiviral” and “antiviral composition” refer to an amount of anti-viral protein associated noble metal nanoparticles that suppress the replication and the spread of viruses, prevent viral attachment, prevent viral replication within the host cell, and/or improving or alleviating the symptoms caused by viral infection. The criteria for effective therapy include lower viral load, lower mortality rate, and/or lower morbidity rate, etc.

As used herein, “derivatives” refers to any derivative of the protein associated noble metal nanoparticles and combinations thereof. Non-limiting example of protein associated noble metal nanoparticles include nanoparticles that associate with one or more proteins via covalent or non-covalent bonding and may include combinations of proteins and even concatamers of protein-nanoparticle-protein, etc., into bi-, tri-, terta-, multimers, oligomers, polymers and the like in two or three-dimensions.

As used herein, “delivering” refers to contacting nanoparticles to a location or target defined as effecting the placement of the nanoparticles attached to, next to, or sufficiently close to the location such that any heat generated by the nanoparticles is transferred to the location. “Delivering” may be targeted or non-targeted as the term “targeted” is used herein.

As used herein, “Nanometer” is 10⁻⁹ meter and is used interchangeably with its abbreviation “nm.”

As used herein, “nanoparticle” refers to defined as a particle having dimensions of from 1 to 5000 nanometers, having any size, shape or morphology. For use with the present invention the nanoparticles are noble metals, such as gold colloid or silver and may be, e.g., nanospheres, nanotubes, nanorods, nanocones and the like.

As used herein, “nanoparticle” refers to one or more nanoparticles. As used herein, “nanoshell” means one or more nanoshells. As used herein, “shell” means one or more shells.

As used herein, “non-tissue” is defined as any material that is not human or animal tissue. As used herein, the term “targeted” refers to the use of protein-protein binding, protein-ligand biding, protein-receptor binding, and other chemical and/or biochemical binding interactions to direct the binding of a chemical species to a specific site.

As used herein, “viral infection” refers to viral invasion of a target cell. When the virus enters the healthy cell, it takes advantage of the host reproduction mechanism to reproduce itself, ultimately killing the cell. As the virus reproduces, newly produced viral progeny infect other cells, often adjacent cells. Some viral genes can also integrate into host chromosome DNA to cause a latent infection via a provirus. The provirus reproduces itself with the replication of the host chromosome, and can bring the infected people into morbidity at any moment if activated by various factors inside and outside the body.

As used herein, “synergic action” refers to a joint protein associated noble metal nanoparticle-drug administration that is more effective than the additive action of merely using any of two or more therapeutics to cure or to prevent viral infection. The synergic effect can increase the efficacy of the antiviral drugs and the protein associated noble metal nanoparticles to avoid or alleviate viral tolerance against any single medicine.

As used herein, “therapeutics” refers the protein associated noble metal nanoparticles whether alone or compounded in a delivery system, whether liquid, solid, gel-like, dried, frozen, resuspended and the like. The protein associated noble metal nanoparticles drug or active agent is conductive to the treatment of viral infection or virus-caused diseases, as taught herein.

The protein associated noble metal nanoparticle antiviral agents of the present invention may be used alone or in combinations with agents that include, but are not limited to antiviral agents, such as the cytokines rIFNα, rIFNβ, and rIFNγ; reverse transcriptase inhibitors, such as AZT, 3TC, ddI, ddC, Nevirapine, Atevirdine, Delavirdine, PMEA, PMPA, Loviride, and other dideoxyribonucleosides or fluorodideoxyribonucleoside; viral protease inhibitors, such as Saquinavir, Ritonavir, Indinavir, Nelfinavir, and VX-478; hydroxyurea; viral mRNA capping inhibitors, such as viral ribovirin; amphotericin B; ester bond binding molecule castanospermine with anti-HIV activity; glycoprotein processing inhibitor; glycosidase inhibitors SC-48334 and MDL-28574; virus absorbent; CD4 receptor blocker; chemokine co-receptor inhibitor; neutralizing antibody; integrase inhibitors, and other fusion inhibitors.

The anti-viral protein-nanoparticles described herein may be used as part of a method and kit for improved antiviral therapy for the treatment of broad viral (including HIV) infection. In addition, the present invention provides a method of joint drug administration aimed at boosting the therapeutic effect, including the use of combination therapy, its derivatives, a second active agent or nutraceutical or dietary supplement, generally provided alone or in combination within a pharmacologically acceptable carrier. An advantage of combination therapy is that it may preclude viral adaptation or mutation that increases its tolerance against each therapeutic alone. Another advantage of combination therapy is that drugs may be provided at lower doses to reduce drug toxicity and enhance the therapeutic index.

It is known that size confinement produces dramatic changes on the physical properties of matter. One of the most well-known effects is the change of optical properties in noble metal nanoparticles with size, known generally as the surface Plasmon resonance effect. Noble metal nanoparticles or nanowires produce changes in the color, i.e., the light scattering by surface plasmons. In the case of transition metals the search for ultra dense magnetic recording devices has promoted the research in nanoparticles. Finite size can have effects on the structural and magnetic order in nanoparticles.

One particular feature of the method of the present invention for noble metal nanoparticles directly functionalized by bovine serum albumin (BSA) is that a near quantitative yield of well-dispersed, protein-conjugated nanoparticles in an aqueous system at ambient conditions is achieved. The physicochemical properties of nanoparticles are determined by their size. High-precision applications require nanoparticles with uniform physicochemical properties, and therefore well-dispersed noble metal nanoparticles are a highly value-added product. Previous bioconjugation techniques typically involved incubation of nanoparticles in the presence of biomolecules at a specific pH. A significant number (25-30%) of the nanoparticles remain unconjugated after incubation, and are separated from the conjugates by centrifugation. This caused the free nanoparticles to coalesce into a waste pellet, and a significant percentage of valuable nanoparticles are lost. In contrast, the method disclosed herein yielded a nearly quantitative yield of protein-conjugated gold nanoparticles.

Certain other beneficial features of the noble-metal nanoparticles synthesized include that the nanoparticles are small (diameter less than 5 nm) and stable, with a narrow, controllable size distribution, and that the nanoparticles are directly functionalized with a globular, macromolecular protein.

Noble metal nanoparticles less than 10 nm in diameter undergo changes in electronic structure due to quantum size effects, and are highly valued for their catalytic, electromagnetic, and optical properties. Current techniques for conjugation of metal nanoparticles with proteins yield unstable complexes. Conjugates are typically stored at 4° C., remain stable only for a period of weeks, and the nanoparticles have a strong propensity to aggregate and coalesce upon heating or if the water content is evaporated. In contrast, our protein-conjugated nanoparticles are stable indefinitely in aqueous solution at ambient conditions. Furthermore, the water content can be evaporated and the product ground into a fine powder without inducing coalescence. The powder is easily redissolved in water, thus making it desirable for storage purposes.

Direct functionalization between the nanoparticle and protein is another desirable feature. Previous methods for linking small nanoparticles with biomolecules required the particle surface to be saturated with a protecting agent or linker molecule prior to conjugation. In the former case, conjugate stability depends on the degree to which the biomolecule displaces the protecting agent. In the latter case, the particles are linked externally to the biomolecule, which can lead to nanoparticle aggregation or formation of multimeric structures among biomolecules. In contrast, the method disclosed herein bypasses protecting agents and linking agents, resulting in nanoparticles that are directly bound to and protected within globular protein. This is a great improvement in terms of product stability, and since the particle surface is not saturated with intermediate agents, our nanoparticles should have free surface area, and are able to interact with external species.

Another important feature of the present invention was the discovery of its antimicrobial and antiviral activity. The nanoparticles of the present invention were used as protein-conjugated silver nanoparticles as antiviral agents, particularly for the inhibition of the HIV-1 virus. Because serum albumin in the body is confined to the circulatory system, the nanoparticles (conjugated directly to serum albumin) have potential for targeted delivery within the bloodstream and may be further targeted using, e.g., anti-gp120, anti-gp120, anti-gp41 or other anti-HIV antibodies. The protein-conjugated nanoparticles could be functionalized with one or more antibodies for enhanced targeting or even for extraction after use, thereby eliminating the unused protein-noble metal nanoparticles.

The method yielded a near-quantitative yield of small (diameter less than 10 nm), well-dispersed, nanoparticles directly conjugated to a macromolecular protein in an aqueous system at ambient conditions. The resulting product was well dispersed, more stable than current bioconjugates, and can be stored indefinitely at ambient conditions as a powder. The small noble-metal nanoparticles are directly conjugated to a globular protein, and possess a large, free surface area for interaction with external species. Furthermore, upon evaporating the aqueous content of the solution, the protein-conjugated nanoparticles form a thin transparent film, ranging in color from light golden brown to dark brown depending on the nanoparticle concentration. These films may possess unique optical properties. Additionally, the protein-conjugated nanoparticles may be used as, e.g., catalysts, biodegradable catalysts and biocompatible metallic catalysts. Alternatively, the nanoparticles can be used in vivo, e.g., as labels for cells and tissues (with a highly electron dense noble metal nanoparticle in cases where physical barriers would filter out larger particles), to separate cells or targets by weight (i.e., centrifugation of cells that became loaded with silver or gold nanoparticles), by charge, by opacity, by reflectivity, for microscopy and the like.

The present invention provides an inexpensive, easily available and convenient method of inhibiting the transmission of the AIDS virus in humans as a result of sexual intercourse. The method relies upon the action of silver nanoparticles which results in a rapid killing action within minutes. These compounds are effective to reduce the infectivity of the AIDS virus and also kill the causative organisms of many other STD's after short exposure. The method of the invention is therefore useful to reduce the immediate risk of AIDS transmission. It also reduces future risk of AIDS transmission by eliminating STD causing organisms which increase the risk of AIDS.

In view of these findings, the invention contemplates a method of inhibiting the transmission of AIDS in humans upon sexual intercourse, by the use of an effective antiviral amount of silver nanoparticles topically applied to a sexual canal of a human prior to or during sexual intercourse. This application may be carried out by introducing a cream or foam into the sexual canal, or by coating the inhibitory composition onto a condom or other device that is inserted into the sexual canal.

The present technology offers the possibility of combining the biocide properties of silver with different materials to produce effective antiviral products preventing AIDS transmission. Some of the potential uses can be: vaginal biocides, disinfectant, biocides, filters, topical antiviral and systemic antiviral. Due to its strong toxicity to a wide range of microorganisms, silver has been used against bacteria and fungi. There is a possibility of using nanotechnology to improve and develop silver nanoparticles to use as biocides in substitution of current products like antibiotics.

The present invention may be used to affect the transmission of AIDS in humans may be slowed or prohibited at or upon sexual intercourse by the use of an effective antiviral amount of silver nanoparticles topically applied to a sexual canal of a human prior to or during sexual intercourse. This application can be carried out by introducing a cream or foam into the sexual canal, or by coating the inhibitory composition onto a condom or other device that is inserted into the sexual canal.

The silver nanoparticles of the present invention take advantage of the biocide properties of silver with different materials to produce effective antiviral products preventing AIDS transmission. Some of the uses are listed in the following paragraphs.

Vaginal Biocides: An agent (e.g., a chemical or antibiotic) that destroys microorganisms in the vagina. Research is being carried out to evaluate the use of rectal and vaginal biocides to inhibit the transmission of sexually transmitted diseases, including HIV. Like today's spermicides, a biocide could be produced in many forms, including; gels, creams, suppositories, films, or in the form of a sponge or a vaginal ring that slowly releases the active ingredient over time, that would give women the power to protect themselves from sexually transmitted diseases (STDs) and AIDS. Around the world women's health and lives are at risk every day because there are too few options in STD protection.

Disinfectant: A chemical which kills viruses and other microorganisms on a nonliving surface.

Biocide: a chemical substance, such as pesticides, which can be herbicides, insecticides, capable of killing different forms of organisms such as viruses used in fields such as agriculture, forestry, and mosquito control. Biocides can also be added to other materials (typically liquids) to protect them from biological infestation and growth.

Filters: to inactivate viral pathogens such as rotavirus in water or any liquid such as human milk from infected women of HIV-1.

Topical antiviral: eye drops or skin creams or gels. Systemic antiviral: providing the nanoparticles systemically by delivering the nanoparticles intravenously, intramuscularly, subcutaneously, intradermally, transdermally, and the like.

The most important characteristic of the present invention is the use of silver nanoparticles as antivirals. The chemical and physical properties that bulk materials exhibit change drastically when the material is in the nanometer range. For this reason there is an increasing appeal in the development of nanomaterials, which can be used in physical, biological, biomedical and pharmaceutical applications.

Changes in physical properties: (1) Increase of the surface area to volume ratio (less silver particles per volume); (2) the size of the particle moving into the realm where quantum effects predominate; and (3) the structure of small nanoparticles fluctuates all the time increasing their catalytic activity.

Transparency: the fact that nanoparticles have dimensions below the critical wavelength of light and the used concentrations are very small, renders them transparent, a property which makes them very useful for applications in cosmetics and therefore also as biocide gels.

Regarding the advantages in viral inhibition, it was found that silver nanoparticles are able to inhibit the HIV-1 virus in concentrations as low as 3 μg/mL. At this concentration, there is no toxicity on MT-2 cells (Human T-cell leukemia cells isolated from cord blood lymphocytes and cocultured with cells from patients with adult T-cell leukemia) and c-magi cells.

Finally, biocides containing silver nanoparticles would work in one of three ways: killing STD and AIDS viruses and bacteria, creating a barrier to block infection, or preventing the virus from replicating after infection has occurred. Ideally, biocides containing silver nanoparticles would be available either with or without spermicide in order to give women the option of becoming pregnant, while still protecting themselves from STDs.

The present invention relates to a method of inhibiting the transmission of Acquired Immunodeficiency Syndrome (AIDS) using silver nanoparticles. The present invention provides an inexpensive, easily available and convenient method of inhibiting the transmission of the AIDS virus in humans as a result of sexual intercourse. The method relies upon the action of silver nanoparticles which results in a rapid killing action within minutes. These compounds are effective to reduce the infectivity of the AIDS virus and also kill the causative organisms of many other STD's after short exposure. The method of the invention is therefore useful to reduce the immediate risk of AIDS transmission. It also reduces future risk of AIDS transmission by eliminating STD causing organisms which increase the risk of AIDS.

The apparatus and method of the present invention is based on the finding that silver nanoparticles, are effective antiviral agents against retroviruses including the AIDS virus. Silver materials had previously been recognized as antibacterial agents useful in treating burns in man and animal. C. L. Fox, Jr., U.S. Pat. No. 3,761,590, relevant portions incorporated herein by reference. Silver in the form of AgSD has also been shown to be effective against certain viruses such as herpes simplex and herpes zoster and against the causative organisms of many STD's including Candida albicans, Treponema pallidum and gonorrhea. U.S. Pat. No. 4,415,565, relevant portions incorporated herein by reference, of Wysor shows further antiviral activity of AgSD against certain RNA viruses, but none of these are retroviruses. Thus, while AgSD is a well studied material, there was no basis to expect that it would have activity against the AIDS retrovirus which has proven so difficult to inhibit or destroy.

According to B. Hanke, U.S. Pat. No. 6,720,006, relevant portions incorporated herein by reference, silver nanoparticles have demonstrated being useful to produce anti-microbial body care products. This opens the possibility of further studies in this area; however no antiviral testing was conducted.

In view of these findings, the invention contemplates a method of inhibiting the transmission of AIDS in humans upon sexual intercourse, by the use of an effective antiviral amount of silver nanoparticles topically applied to a sexual canal of a human prior to or during sexual intercourse. This application can be carried out by introducing a cream or foam into the sexual canal, or by coating the inhibitory composition onto a condom or other device that is inserted into the sexual canal.

There is a lack of studies analyzing the health impact of silver nanoparticles inside the human body. However, there is evidence that silver nanoparticles in proper concentrations are not dangerous for external use, U.S. Pat. No. 6,720,006, relevant portions incorporated herein be reference, and many references about the use of colloidal silver for health purposes.

There are several articles about the bactericidal properties of ionic silver. However, these articles focus on the known properties of silver nanoparticles (I. Sondi, B. Salopek-Sondi, J. Colloid Interface Sci. 275, 177-182 (2004) relevant portions, methods of manufacture and preparation, incorporated herein be reference) against bacteria.

Dosage Forms. The silver nanoparticles may also be administered, e.g., parenterally, intraperitoneally, intraspinally, intravenously, intramuscularly, intravaginally, subcutaneously, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, poly-ol (for example, glycerol, propylene glycol, and liquid polyethylene glycol, and the like), suitable mixtures thereof, and vegetable oils.

The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the therapeutic compound) plus any additional desired ingredient from a previously sterile-filtered solution thereof.

The silver nanoparticles may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied as will be known to the skilled artisan. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

Aqueous compositions of the present invention comprise an effective amount of the noble metal nanoparticle, nanofibril or even a nanoshell or chemical composition of the present invention dissolved and/or dispersed in a pharmaceutically acceptable carrier and/or aqueous medium. The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous composition that contains an effective amount of the nanoshell composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts may be prepared in water suitably mixed with a surfactant, such as hydroxypropylcellulose. Dispersions may also be prepared in glycerol, liquid polyethylene glycols, and/or mixtures thereof and/or in oils. Under ordinary conditions of storage and/or use, these preparations contain a preservative to prevent the growth of microorganisms.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle that contains the basic dispersion medium and/or the required other ingredients from those enumerated above. In the case of sterile powders for the preparation of sterile injectable solutions, the preferred methods of preparation are vacuum-drying and/or freeze-drying techniques that yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like may also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media that may be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations that are suitable for other modes of administration include vaginal suppositories and/or suppositories. A rectal suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup or elixir may contain the active compounds sucrose as a sweetening agent methyl and/or propylparabens as preservatives, a dye and/or flavoring, such as cherry and/or orange flavor.

Substrates. The substrate of the compositions of the present invention may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder constitutes a finely divided (milled, micronized, nanosized, precipitated) form of an active ingredient or additive molecular aggregates or a compound aggregate of multiple components or a physical mixture of aggregates of an active ingredient and/or additives. Such substrates may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin.

It should be emphasized that a substrate need not be a solid material, although often it will be a solid. For example, the encapsulation coat on the substrate may act as a solid “shell” surrounding and encapsulating a liquid, semi-liquid, powder or other substrate material. Such substrates are also within the scope of the present invention, as it is ultimately the carrier, of which the substrate is a part, which must be a solid. The silver nanoparticles of the present invention may be used as a topical cream against HIV and other retroviruses. The cream described may also be used in condoms.

Excipients. The silver nanoparticle pharmaceutical compositions of the present invention may include optionally one or more additives, sometimes referred to as additives. The excipients may be contained in an encapsulation coat in compositions, which include an encapsulation coat, or can be part of the solid carrier, such as coated to an encapsulation coat, or contained within the components forming the solid carrier. Alternatively, the excipients may be contained in the pharmaceutical composition but not part of the solid carrier itself. Suitable excipients are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

Sterile intravenous (iv) solutions such as saline may be effective in reducing virus load and slowing down the onset of immunodeficiency. Surgeons who also use saline washes in cleansing a particular area in the operating field may find it useful. The silver nanoparticles may be used alone or in conjunction with a liposome. These forms could be reconstituted in the form of mouthwash with the silver nanoparticles alone or in conjunction with antifungal reagents. An inhalant form alone or in conjunction with pentamidine. The use of silver nanoparticles in tablet form to be taken orally. The oral use of the liposomal form would have to be given in a time release capsule to avoid lipase degradation.

Buffered ophthalmic solution—for patents suffering from HIV associated retinitis. The buffering is necessary due to pH changes the silver nanoparticles may cause. Highly concentrated solution for intramuscular injection—would facilitate treatment of needle stick injuries of health care workers. In this regard use of DMSO as solvent would give extremely fast penetration delivering high concentrations of silver nanoparticles to a small area. Suppository form—for chemoprevention in homosexuals because the major sites of infection are the large intestine and rectum. Chemo-preventative Vaginal douche and cream—the douche may be of use in a pre-sexual exposure in a standard acetic acid solution. The cream may be mixed with 9-nonoxynol spermicide to use in conjunction with birth control. Vaginal sponge—this could be used by prostitutes so that silver nanoparticles would be time-released over several hours with nonoxynol. Gloves lined with silver nanoparticles may help surgeons and other health care workers dealing heavily with blood and bodily fluids. The use of silver nanoparticles in liquid soap in combination with anti-bacterial agents may be useful in hospitals and research institutions. Although this would probably be no more effective than plain anti-bacterial soap, the employees and hospital insurance companies would appreciate it.

In operation, the present invention takes advantage of the newly discovered physicochemical properties of nanoparticles that strongly dependent upon their interactions with capping agent molecules.¹⁷ The inventors tested silver nanoparticles possessing three different types of capping agents: carbon, poly (N-vinyl-2-pyrrolidone) (PVP), and bovine serum albumin (BSA). Carbon-coated nanoparticles were obtained from Nanotechnologies, Inc., and used without further treatment (see, e.g., www.nanoscale.com/products_silver.asp). The carbon matrix serves to prevent coalescence during synthesis, but the inventors have found that there is only a weak attraction between the carbon and the nanoparticles. As a result, these nanoparticles possess nearly free surfaces. PVP coated nanoparticles were synthesized by a modified polyol process in glycerin. PVP is a linear polymer and stabilizes the nanoparticle surface via bonding with the pyrrolidone ring. Silver nanoparticles directly conjugated to BSA protein molecules were synthesized in aqueous solution. BSA stabilizes nanoparticles via direct bonding with thiol-bearing cysteine residues, and provides steric protection due to the bulkiness of the protein.

The particles of the present invention were made and used as follows. HIV-1 strains and cell lines. HIV-1 and cells: HIV-1IIIB laboratory strain of HIV-1 an X4 wild type (wt) virus was obtained through the AIDS Research and Reference Reagent Program, Division of AIDS, NIAID, NIH. CD4+ MT-2 cell line was obtained from the American Type Culture Collection. The cMAGI HIV-1 reporter cells were a generous gift from Dr. Phalguni Gupta from the University of Pittsburgh. All other reagents used were of the highest quality available.

Cell culture and virus propagation. cMAGI cells were cultured in DMEM Dulbecco's Modified Eagle Medium (DMEM) (1×) liquid without sodium phosphate and sodium pyruvate. The medium contained 4,500 mg/L D-glucose and L-glutamine (Invitrogen, Paisley, UK), with 10% fetal calf serum (FCS), 0.2 mg/mL geneticin (G418), and 0.1 μg/mL puromycin. MT-2 cells were cultured in RPMI 1640, containing 10% fetal calf serum (FCS) and antibiotics.

HIV-1IIIB primary clinical isolates were propagated by subculture in MT-2 and cMAGI cells. HIV-1IIIB was reproduced according to the DAIDS Virology Manual for HIV Laboratories, version 1997, compiled by Division of AIDS of the National Institute of Allergies and Infectious Diseases and the National Institute of Health, and Collaborators. Aliquots of cell-free culture viral supernatants were used as viral inocula. Carbon coated silver nanoparticles. Carbon coated silver nanoparticles tested in this study were obtained from Nanotechnologies, Inc. (Austin, Tex.) and used without further treatment. Electron microscopy of the particles revealed that they have an average diameter of 22 nm with a standard deviation of 18 nm.

PVP coated silver nanoparticles. These silver nanoparticles were synthesized by a modified polyol method using glycerine as both reducing agent and solvent. Silver sulfate (Ag₂SO₄, reagent grade) and poly (N-vinyl-2-pyrrolidone) (PVP-K30, MW=40,000) were purchased from Sigma Aldrich and propylene glycol (Glycerin, >99%) was purchased from Fischer Chemicals, all the materials were used without any further treatment. Briefly, we added 0.2 g of PVP to a round bottom flask following by the addition of 30 mL of glycerin. Once PVP was dissolved, we increased the temperature to 140° C. After 30 minutes we added 2 mL of 0.015M Ag₂SO₄ and left to react for 1 h. Nanoparticle size distribution was obtained from HAADF images, based on measurement of 170 particles. The synthesized nanoparticles exhibited an average size of 6.49±2.29 nm.

BSA coated silver nanoparticles. Silver nanoparticles directly conjugated to bovine serum albumin (BSA) protein molecules were produced using a modified version of our previously reported synthesis method.¹ Silver nitrate (AgNO₃, 0.945 M), sodium borohydride (NaBH₄, 99%) and 200 proof spectrophotometric-grade ethanol were purchased from Aldrich. Bovine serum albumin (BSA) was purchased from Fisher and was used without further treatment. Briefly, sodium borohydride was added to an aqueous solution of silver nitrate and BSA under vigorous stirring. The molar ratio of Ag+:BSA was 28:1, and the molar ratio of Ag+:BH₄— was 1:1. The reaction volume was 40 mL, and contained 13.50 gmol BSA. The reaction was allowed to proceed for 1 h, and the product was purified by precipitation at −5° C., followed by cold ethanol filtration. Nanoparticle size distributions were obtained from HAADF images, based on measurement of 500 particles. The BSA-conjugated silver nanoparticles exhibited a bimodal size distribution, with ˜90% being 2.08±0.42 nm, and ˜10% being 6.17±1.72 nm in diameter.

Serum albumin is a globular protein, and is the most-abundant protein in blood plasma. It transports hydrophobic molecules through the blood, and aids in regulating blood pH. Bovine serum albumin (BSA) is a single polypeptide chain composed of 583 amino acid residues2. The structure of human serum albumin, a structural analogue to BSA, has been determined by Curry, et al., from X-ray diffraction.³¹ The space group is C121, with cell constants a=189.18 Å, b=38.96 Å, c=96.40 Å, and β=105.31°. Several residues of BSA have sulfur-, oxygen-, and nitrogen-bearing groups that can stabilize the nanoparticle surface. The strongest interactions with silver likely involve the 35 thiol-bearing cysteine residues.

The capacity of silver nanoparticles to inhibit HIV-1 infectivity was determined by testing against CD4+ MT-2 cells and cMAGI HIV-1 reporter cells. The cytopathic effects of the viral infection of CD4+ MT-2 cells were analyzed by optical microscopy assessment of syncytium formation as described elsewhere,¹⁸ as well as by the HIV-1 infection of cMAGI cells using the Blue Cell Assay.¹⁹-21 All data was obtained by analysis of duplicate samples by two independent observers.

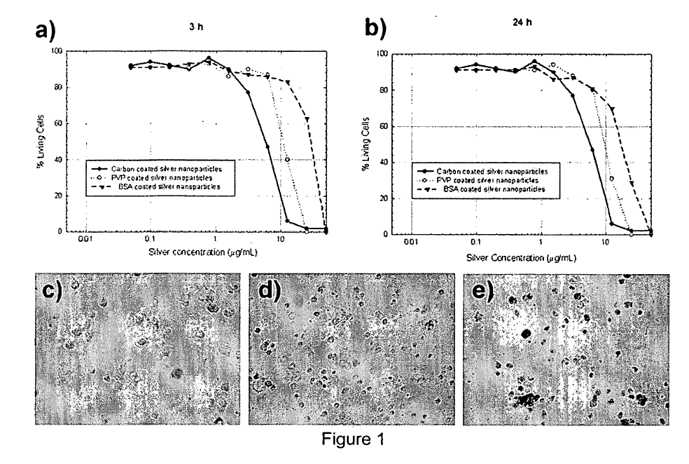

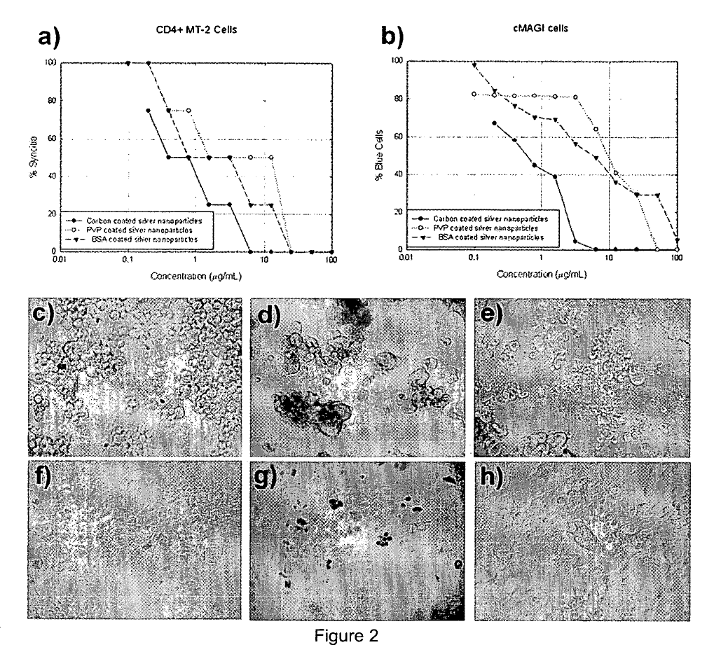

For all the silver nanoparticle preparations tested, the inventors found a dose-dependant inhibition of HIV-1 infectivity with an IC50 where only moderate toxicity was observed in the cultured cells, as shown in FIGS. 1 and 2. At concentrations above 25 μg Ag/mL, the viral infectivity was reduced to an extent that it could not be detected by syncytium formation.

FIGS. 1a through 1e document the toxicity of silver nanoparticles in MT-2 cells. The toxicity of the nanoparticle preparations against MT-2 cells was determined using the Trypan Blue exclusion assay38. In all cases, the initial concentration of silver nanoparticles was 50 μg/mL and sequential two-fold dilutions were made and mixed with 2×105 MT-2 cells. The samples were incubated at 37° C., and the cells were evaluated via optical microscopy after 1a (3 h) and 1b (24 h) of exposure to silver nanoparticles. Briefly, an aliquot of the cell suspension was diluted 1:1 (v/v) with 0.4% Trypan Blue and the cells were counted using a haemocytometer. Viability was expressed as the percentage of number of unstained treated cells to that of the total number of cells. FIGS. 1c through 1e are optical microscopy images of MT-2 cells after 24 h of exposure to carbon coated silver nanoparticles. The concentration of silver was 3, 12 and 50 μg/mL, respectively (10× magnification).

FIGS. 2a through 2h shows the inhibition of HIV-1 by silver nanoparticles. RPMI medium only or containing varying concentrations of silver nanoparticles were mixed with samples 105 TCID50 of HIV-1IIIB cell free virus. The highest concentration of silver nanoparticles used was 100 μg/mL. After 30 seconds, sequential 2-fold dilutions of the solutions were added to cultures of target cells (2×10⁵ MT-2 and 2×10⁵ cMAGI HIV-1 reporter cells with 0.2-0.5 multiplicity of infection (m.o.i) of HIV-1IIIB virus). Each dilution was exposed to four replicate wells. After that, the cells were incubated in a 5% CO₂ humidified incubator at 37° C. for 3-5 days. FIG. 2a, Assessment of HIV-1 mediated syncytium formation was performed for the MT-2 cells, while for FIG. 2b, cMAGI cells, the percentage of transmission was estimated as follows: the number of blue-stained cells obtained from the supernatant of each of the tested wells was divided by the number of blue-stained cells obtained from the culture supernatant in the well of the positive control. Images of FIG. 2c, MT-2 cells negative control, FIG. 2d, MT-2 cells positive control and FIG. 2e, HIV-1 treated MT-2 cells where the virus was previously exposed to carbon coated silver nanoparticles at a concentration of 6 μg/mL. Images of FIG. 2f, cMAGI cells negative control, FIG. 2g, cMAGI cells positive control and FIG. 2h, HIV-1 treated cMAGI cells where the virus was previously exposed to carbon silver nanoparticles at a concentration of 6 μg/mL (10× magnification).

The observed trends in toxicity and HIV-1 inhibition can be explained in terms of the interaction of the silver nanoparticles with the various capping agents. BSA- and PVP-protected nanoparticles exhibit lower toxicities because the highly reactive particle surface is directly bound to and encapsulated by the capping agent. In contrast, the carbon-coated nanoparticles have a greater inhibitory effect against the virus due to their essentially free surface area.

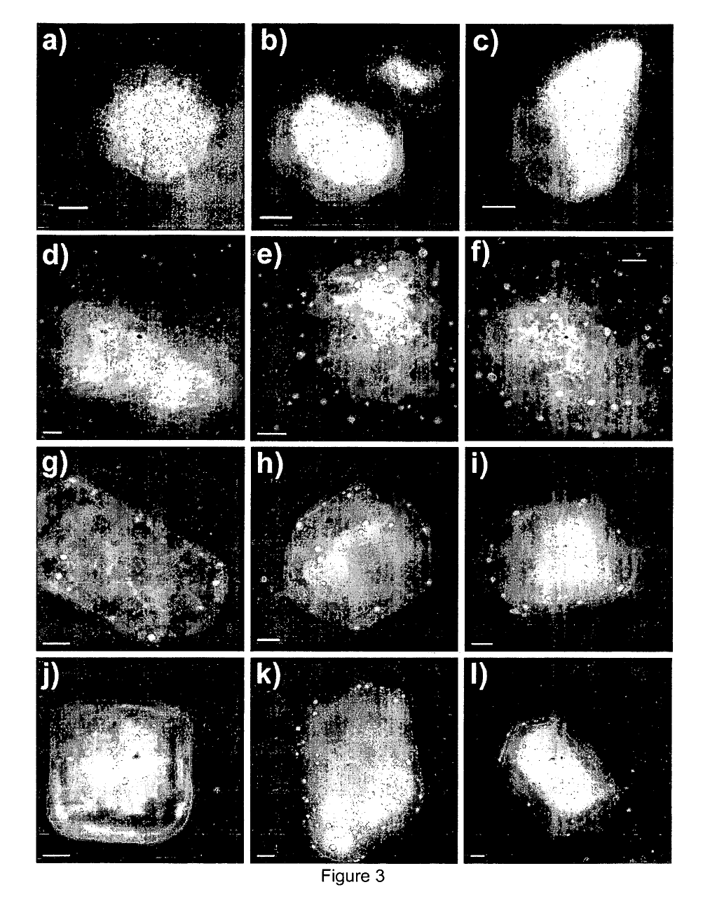

High angle annular dark field (HAADF) scanning transmission electron microscopy was employed to elucidate the mechanism by which silver nanoparticles reduce the infectivity of HIV-1 to undetectable levels. HAADF images are primarily formed by electrons that have undergone Rutherford backscattering. As a result, image contrast is related to differences in atomic number^{22,23} and intensity varying as ˜Z2. Therefore, image contrast is related to composition. As a good approximation, lighter elements appear dark and heavier elements appear bright. For these reasons, even very small (˜1 nm) silver nanoparticles on the virus were clearly observed, as seen in FIG. 3d through 31.

FIGS. 3a through 31 are HAADF images of the HIV-1 virus. Briefly, FIGS. 3a-3c, HIV-1 virions without nanoparticle treatment; 3d-3f are HIV-1 virions exposed to carbon-coated silver nanoparticles; and 3g-3l are HIV-1 virions exposed to BSA-conjugated silver nanoparticles. Two distinct types of morphology were observed. Many virions are nearly spherical in shape with a porous or textured appearance, such as a. the inventors attribute this morphology to the immature form of the virus. The second type of virion exhibits a faceted morphology, which the inventors ascribe to the mature form of the virus. The majority of faceted virions appear to possess an icosahedral symmetry, as seen in 3b and 3h. Many other faceted virions seem to be nearly icosahedral, such as 3i and 3l. More exotic faceted morphologies were also observed, such as 3g, 3j, and 3k. The samples shown in FIG. 3a through 3l were prepared for electron microscopy as follows: 105 TCID50 samples of HIV-1IIIB cell free virus were treated with solutions of the different silver nanoparticles at a concentration of 100 μg/mL. After 30 seconds, a 10 μL droplet was deposit on a carbon coated nickel TEM grid and exposed to a 2.5% solution of PBS/glutaraldehyde vapors for 30 minutes. Microscopy was done using a JEOL 2010-F TEM equipped with an Oxford EDS unit, at an accelerating voltage of 200 kV and operated in scanning mode using an HAADF detector. Scale bars: 20 nm.

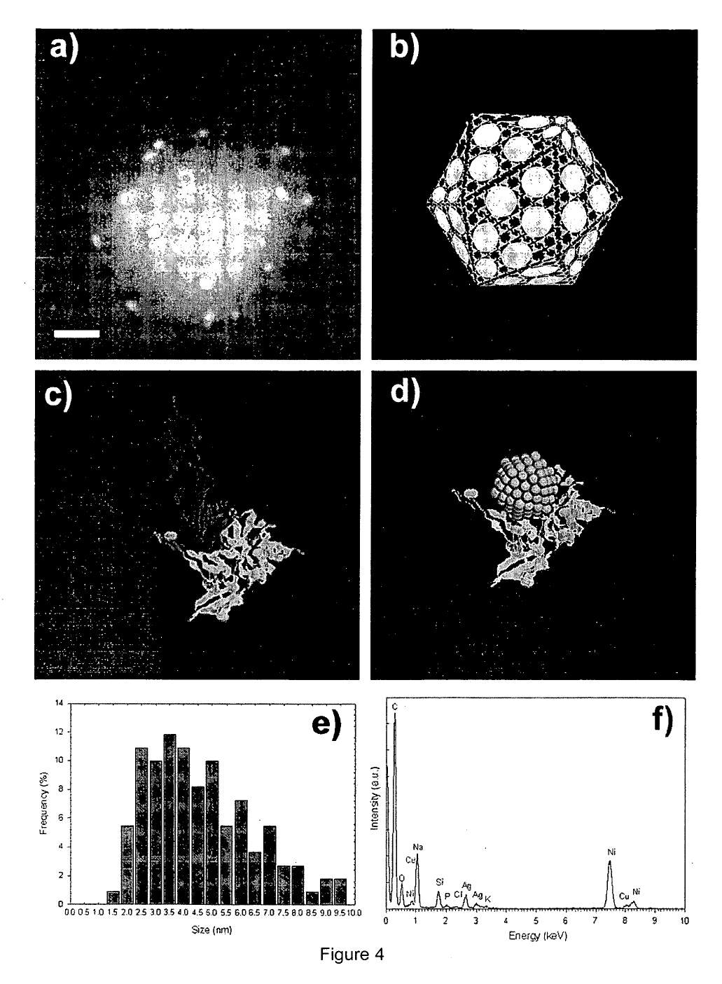

The presence of silver was independently confirmed by Energy Dispersive X-ray Spectroscopy (EDS), seen in FIG. 4f. The sizes of nanoparticles bound to the virus (FIG. 4e) were exclusively within the range of 1-10 nm.

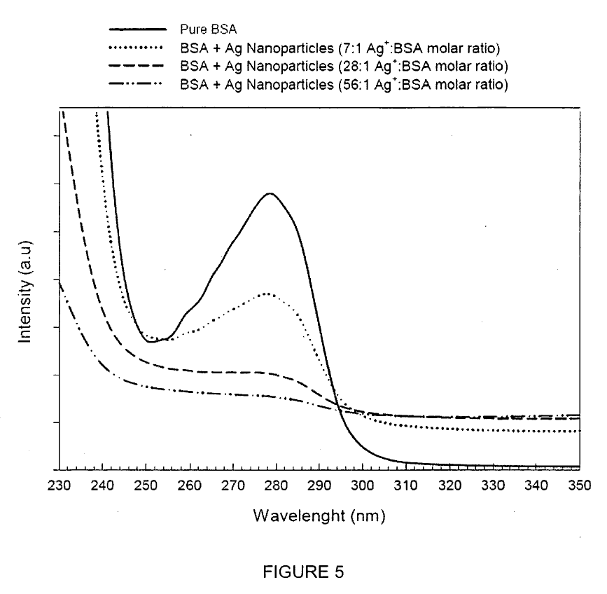

FIGS. 4a through 4f summarize the specific interaction of silver nanoparticles with HIV-1 and provide models that support the same. FIG. 4a is an HAADF image of an icosahedral virus with regular spatial relationships clearly observed among attached nanoparticles (Scale bar: 20 nm). FIG. 4b is a structural model of an icosahedral HIV-1 virion, after Nermut, et al.,²⁵ the crcles represent the positions of glycoprotein gp120. FIG. 4c is a tertiary structure of gp120 determined by Kwong, et al.³⁹ (PDB 1GC1)(disulfides are displayed as space-filled atoms). FIG. 4d is a schematic representation of a silver nanoparticle binding with the gp120 subunit of the HIV-1 envelope glycoprotein. FIG. 5e is a graph that shows the composite size distribution of silver nanoparticles bound to the HIV-1 virus, derived from all tested preparations. FIG. 4f is an EDS analysis of image a confirming the presence of Ag. The C signal comes from both the TEM grid and the virus, O, and P are from the virus, and Na, Cl, and K are present in the culture medium. Ni and Si come from the TEM grid, while Cu is attributed to the sample holder.

The global symmetry of HIV-1, or lack thereof, has been a subject of great debate.^{24,25} Previous electron microscopy studies have led some researchers to conclude that mature virions have global icosahedral symmetry while the immature virions tend to be more spherical.^{26,28} FIG. 3 shows images of HIV-1 morphology obtained using High Angle Annular Dark Field (HAADF) electron microscopy. These results confirm that the mature form of the virus preferentially assumes an icosahedral symmetry. Other virus morphologies observed via HAADF are also presented in FIG. 3, and reflect the complexity of this virus. Even in the case of immature viruses, shown in FIGS. 3a and 3d trough 3f, the inventors found evidence of local order, as observed in previous studies.^{29,30}

As can be clearly seen in FIG. 4a, the nanoparticles are not randomly attached to the virus, but are located at specific positions, with regular spatial relationships observed among groups of three particles. Nermut and coworkers²⁵ developed a structural model for HIV-1 based on experimental data that supports the icosahedral symmetry in mature forms of the virus. Their model is composed of a regular icosahedron with three gp120 knobs per face, as shown in FIG. 4b. These observations are clear in light of this model, and the inventors conclude that the nanoparticles are interacting preferentially with the gp120 subunit of the HIV-1 envelope glycoprotein. The inventors propose, but are not bound in theory to, the observation that the interaction between the glycoprotein and the nanoparticle is facilitated by the disruption of exposed disulfide bonds, and formation of Ag—S bonds.

The present inventors had previously demonstrated the ability of gold nanoparticles to disrupt disulfide bonds of BSA protein.³¹ Briefly, the UV-visible absorption spectrum of BSA exhibits a maximum at 278 nm due to the aromatic residues tryptophan and tyrosine and the disulfide bonds in the protein.³² The inventors measured the absorbance spectrum of BSA conjugated with different concentrations of gold nanoparticles, and observed that the absorbance at 278 nm decreases with increased gold loading. Infrared spectroscopy revealed that the aromatic residues remain intact, and Raman spectroscopy indicated a loss of S—S stretching at 508 cm-1. Therefore, it was proposed that the decreased absorbance reflects the disruption of disulfide bonds.

To confirm that silver nanoparticles have the same capability, we obtained equivalent UV-visible absorbance spectra for BSA conjugated with different concentrations of silver nanoparticles (See FIG. 5). Briefly, UV-visible absorbance spectra for BSA conjugated with different concentrations of silver nanoparticles. Absorption spectroscopy was performed for pure BSA and silver-conjugated BSA with initial Ag+:BSA molar ratios of 7:1, 28:1, and 56:1. The intensity of absorbance at 278 nm decreases with increased silver loading, as in the case of gold-conjugated BSA. The absorbance at 278 nm also decreases with increased silver loading. Because both products are similar in terms of size and stability, we conclude that the phenomena are congruent, and that the decreased absorbance reflects the disruption of disulfide bonds as Ag—S bonds are formed at the nanoparticle surface.

A comparable mechanism is proposed for the interaction of silver nanoparticles with HIV-1 gp120 glycoproteins. The gp120 subunit of the viral envelope glycoprotein has several disulfide bonds. Specifically, Leonard and coworkers³³ reported nine disulfide bonds, three of which are located in the vicinity of the CD4 binding domain. The inventors propose, but are not bound in theory to, the observation that the silver nanoparticles interact selectively with the exposed disulfide bonds to form Ag—S bonds.

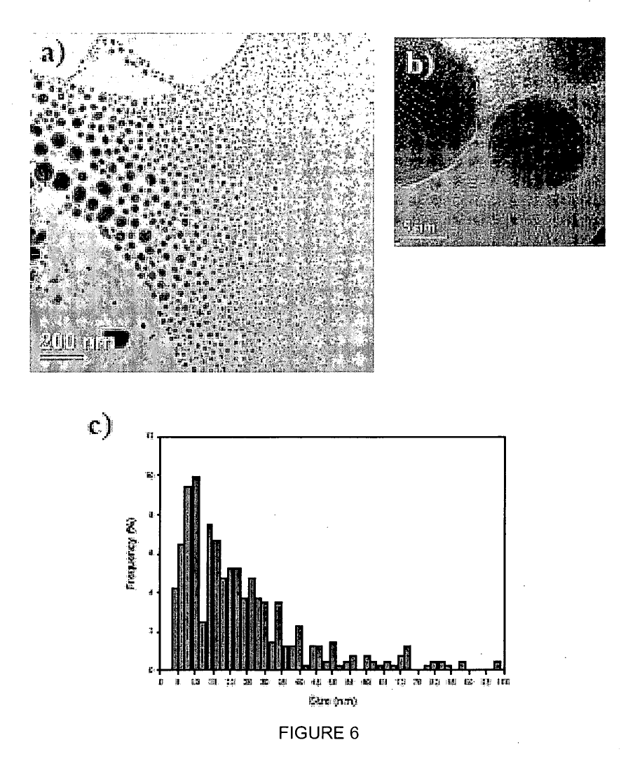

As noted previously, only nanoparticles within the range of 1-10 nm were observed to bind with the virus. For the case of carbon-coated nanoparticles, a large portion of the population is greater than 10 nm in diameter (for a complete size distribution histogram and electron microscopy images of the silver nanoparticles preparations, See FIGS. 6-8), so the interaction of nanoparticles with gp120 would seem to be size-dependent.

FIGS. 6A-6C are a transmission electron micrograph, a High Angle Annular Dark Field image and a particle size distribution histogram, respectively, of carbon-coated silver nanoparticles of the prior art.

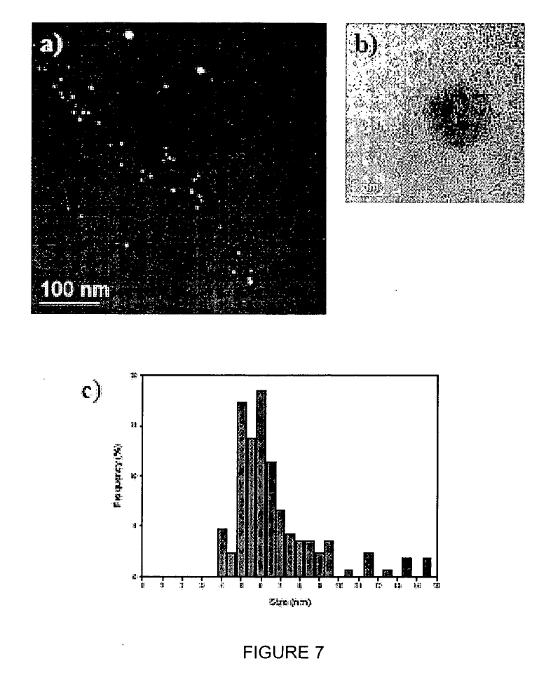

FIGS. 7A-6C are a transmission electron micrograph, a High Angle Annular Dark Field image and a particle size distribution histogram, respectively, of PVP-coated silver nanoparticles developed by the present inventors.

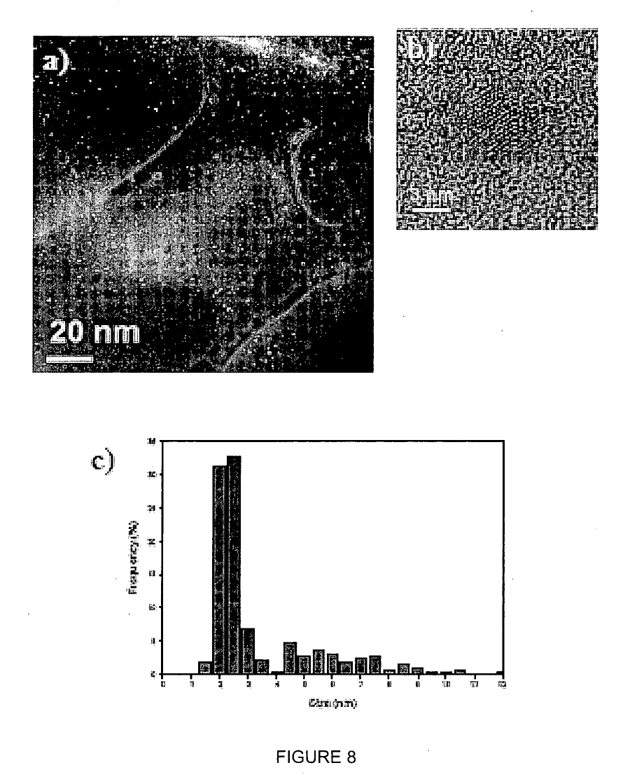

FIGS. 8A-6C are a transmission electron micrograph, a High Angle Annular Dark Field image and a particle size distribution histogram, respectively, of the Albumin-coated silver nanoparticles of the present invention.

As noted, the interaction of nanoparticles with gp120 would seem to be size-dependent, which may be attributed to the size of the glycoprotein knobs (diameter ˜14 nm²⁵), but this phenomenon may also a function of the unique properties and increased reactivity of nanoparticles in this size range. TEM studies on small (<5 nm) metal particles³⁴⁻³⁶ have demonstrated that small excitations may be sufficient to induce structural fluctuations, and that the rates of such fluctuations increase with decreasing the size of the nanocrystal. The total potential surface energy for the different morphologies of a nanoparticle consists of several minima, and the barriers between these states are small enough (˜kT) so thermal fluctuations provide enough energy to produce changes in morphology.³⁷ This highly fluxional character increases the reactivity of the nanoparticles.

Direct conjugation of nanoparticles to plasma proteins could provide a strategy for restricting nanoparticles to circulation within the bloodstream. Due to its large size and negative charge, serum albumin is generally confined to the vasculature. Since HIV-1 replicates within the bloodstream, nanoparticles directly attached to such molecules have potential for targeted antiviral delivery. Conjugation of globular proteins with antibodies is common, and functionalizing protein-nanoparticle conjugates with virus-specific antibodies could provide enhanced targeting.

In conclusion, the inventors have demonstrated that silver nanoparticles inhibit HIV-1 by specific interactions with the gp120 subunit of the viral envelope glycoprotein. For the sake of clarity and not intending to be bound in theory to or in any way restrict the claims of the present invention, the inventors propose that the interaction occurs because the silver nanoparticles disrupt exposed disulfide bonds to form Ag—S linkages. Due to this preferential interaction with gp120, silver nanoparticles block the virus from binding with host cells, thereby preventing infection. The nanoparticles bound to the virus were exclusively within the range of 1-10 nm, and the inventors believe this is a function of the increased reactivity of nanoparticles of this size. Because the proposed inhibitory mechanism involves disruption of disulfide bonds, it is likely that other transition-metal nanoparticles on the order of 1-10 nm in diameter will exhibit antiviral activity. The flexibility of nanoparticle preparation methods, the multitude of possible functionalization techniques, and the facile incorporation of nanoparticles into a variety of media open a new field of antiviral research.

Noble metal nanoparticle-protein complex may be added slowly to an aqueous solution of polyvinylpyrrolidone and mixed well. Next, No. 25-30 mesh sugar spheres are coated with the noble metal nanoparticle-protein complex drug solution in a fluid bed granulator. The drug containing pellets were dried, and a seal coat of Opadry Clear and the inner mixed release coating applied to the active particles by spraying a solution of ethylcellulose and diethyl phthalate in 98/2 acetone/water. The outer coating of a blend of ethylcellulose and HPMCP plasticized with diethyl phthalate was sprayed onto the active particles having the inner coating to produce modified release profile beads. These beads are filled into hard gelatin capsules using capsule filling equipment to produce noble metal nanoparticle-protein complex mini-tabs, 2.5, 5.0, 7.5, 8.0, 12.0, 16.0 and 20.0 mg.

A capsule for immediate release of a first active and extended release of a second active in an enveloped formulation, in a single capsule. The noble metal nanoparticle-protein complex may be freeze-sprayed, lyophilized, vacuum dried, heat dried, heat-vacuum dried, etc. to form a powder following isolation and purification. The following is an example of the Noble metal nanoparticle-protein complex as part of a capsule. The skilled artisan will recognize that these formulations may be prepared in mixed immediate, intermediate and long-term or extended release.

Noble metal nanoparticle-protein complex

Talc

Povidone K-30

Maltodextrin MD-40

Syloid

Stearic Acid

Capsule 1

A formulation for release in a gelcap:

Noble metal nanoparticle-protein complex

Talc

Povidone K-30

Maltodextrin MD-40

Syloid

Stearic Acid

Gelcap 1

A formulation for release of the active in a suppository:

Noble metal nanoparticle-protein complex

Talc

Povidone K-30

Maltodextrin MD-40

Syloid

Stearic Acid

beeswax/glycerol

An effervescent tablet for immediate release of a first active and extended release of a second active in an enveloped formulation, in an effervescent tablet:

Noble metal nanoparticle-protein complex

Talc

Povidone K-30

Maltodextrin MD-40

Stearic Acid

Sodium bicarbonate

For immediate release in a caplet:

Noble metal nanoparticle-protein complex

Talc

Povidone K-30

Maltodextrin MD-40

Stearic Acid

Compressed into a Caplet

In a liquid composition, the present invention may be provided as follows:

Noble metal nanoparticle-protein complex

Excipient

Flavorant

Biocompatible Isotonic liquid (e.g., saline)

Buffer

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Coplan, P. M., Mitchnick, M. & Rosenberg, Z. F. Regulatory challenges in microbicide development. Science 304, 1911-1912 (2004).
2. Lederman, M. M. et al. Prevention of vaginal SHIV transmission in rhesus macaques through inhibition of CCR5. Science 306, 485-487 (2004).
3. Collier, C. P., Vossmeyer, T. & Heath, J. R. Nanocrystal superlattices. Annu. Rev. Phys. Chem. 49, 371-404 (1998).
4. Murray, C. B., Kagan, C. R. & Bawendi, M. G. Synthesis and characterization of monodisperse nanocrystals and close-packed nanocrystal assemblies. Annu. Rev. Mat. Sci. 30, 545-610 (2000).
5. Schaaff, T. G. et al. Isolation of smaller nanocrystal Au molecules: Robust quantum effects in optical spectra. J. Phys. Chem. B 101, 7885-7891 (1997).
6. Nam, J.-M., Thaxton, C. S. & Mirkin, C. A. Nanoparticle-based bio-bar codes for the ultrasensitive detection of proteins. Science 301, 1884-1886 (2003).
7. Elghanian, R., Storhoff, J. J., Mucic, R. C., Letsinger, R. L. & Mirkin, C. A. Selective Colorimetric Detection of Polynucleotides Based on the Distance-Dependent Optical Properties of Gold Nanoparticles. Science 277, 1078-1081 (1997).
8. Tkachenko, A. G. et al. Multifunctional Gold Nanoparticle-Peptide Complexes for Nuclear Targeting. J. Am. Chem. Soc. 125, 4700-4701 (2003,).
9. Chan, W. C. W. et al. Luminescent quantum dots for multiplexed biological detection and imaging. Current Opinion in Biotechnology 13, 40-46 (2002).
10. Hirsch, L. R. et al. Nanoshell-mediated near-infrared thermal therapy of tumors under magnetic resonance guidance. PNAS 100, 13549-13554 (2003).
11. Gupta, A. & Silver, S. Silver as biocide:Will resistance become a problem? Nat. Biotechnol. 16, 888 (1998).
12. Feng, Q. L. et al. Mechanistic study of the antibacterial effect of silver ions on Escherichia coli and Staphylococcus aureus. J. Biomed. Mater. Res. 52, 662-668 (2000).
13. Sondi, I. & Salopek-Sondi, B. Silver nanoparticles as antimicrobial agent: A case study on E. coli as a model for Gram-negative bacteria. J. Colloid Interface Sci. 275, 177-182 (2004).
14. Morones, R. et al. The bactericidal effect of silver nanoparticles. Submitted to Nanotechnology (2004).
15. Berger, E. A., Murphy, P. M. & Farber, J. M. Chemokine receptors as HIV-1 coreceptors: Roles in Viral Entry, Tropism, and Disease. Annu. Rev. Immunol. 17, 657700 (1999).
16. Sun, J. et al. Syncytium formation and HIV-1 replication are both accentuated by purified influenza and virus-associated neuraminidase. J. Biol. Chem. 277, 98259833 (2002).
17. Bradley, J. in Clusters and Colloids: From Theory to Applications 459-536 (VCH, Weinheim, 1994).
18. Harada, S., Koyanagi, Y. & Yamamoto, N. Infection of HTLV-III/LAV in HTLV-I-carrying cells MT-2 and MT-4 and application in a plaque assay. Science 229, 563-566 (1985).
19. Deng, H. et al. Identification of a major co-receptor for primary isolates of HIV1. Nature 381, 661 (1996).
20. Chackerian, B., Long, E., Luciw, P. & Overbaugh, J. Human immunodeficiency virus type 1 coreceptors participate in postentry stages in the virus replication cycle and function in simian immunodeficiency virus infection. J. Virol. 71, 3932-3939 (1997).
21. Borkow, G. et al. Adenovirus Expressing a Bioluminescence Reporter Gene and cMAGI cell Assay for the Detection of HIV-1. Virus Genes 29, 257-265 (2004).
22. Howie, A., Marks, L. D. & Pennycook, S. J. New Imaging Methods for Catalyst Particles. Ultramicroscopy 8, 163-174 (1982).
23. James, E. M. & Browning, N. D. Practical aspects of atomic resolution imaging and analysis in STEM. Ultramicroscopy 78, 125-139 (1999).
24. Wilk, T. & Fuller, S. D. Towards the structure of the human immunodeficiency virus: divide and conquer? Current Opinion in Structural Biology 9, 231-243 (1999).
25. Forster, M. J., Mulloy, B. & Nermut, M. V. Molecular modelling study of HIV p17gag (MA) protein shell utilising data from electron microscopy and X-ray crystallography. J. Mol. Biol. 298, 841-857 (2000).
26. Garnier, L., Bowzard, J. & Wills, J. Recent advances and remaining problems in HIV assembly. AIDS 19, S5-S16 (1998).
27. Nermut, M. V., Grief, C., Hashmi, S. & Hockley, D. J. Further evidence of icosahedral symmetry in human and simian immunodeficiency virus. AIDS Res. Hum. Retroviruses 9, 929-938 (1993).
28. Briggs, J. A., Wilk, T., Welker, R., Krausslich, H.-G. & Fuller, S. D. Structural organization of authentic, mature HIV-1 virions and cores. The EMBO Journal 22, 1707-1715 (2003).
29. Fuller, S. D., Wilk, T., Gowen, B. E., Krausslich, H.-G. & Vogt, V. M. Cryoelectron microscopy reveals ordered domains in the immature HIV-1 particle. Current Biology 7, 729-738 (1997).
30. Nermut, M. V. et al. Further Evidence for Hexagonal Organization of HIV gag Protein in Prebudding Assemblies and Immature Virus-like Particles*1. J. Struct. Biol. 123, 143-149 (1998).
31. Burt, J. L., Gutierrez-Wing, C., Miki-Yoshida, M. & Jose-Yacaman, M. J. Noble-Metal Nanoparticles Directly Conjugated to Globular Proteins. Langmuir 20, 11778-11783 (2004).
32. Peters, T., Jr. in All About Albumin: Biochemistry, Genetics, and Medical Applications 9-75. (Academic Press, San Diego, 1996).
33. Leonard, C. et al. Assignment of intrachain disulfide bonds and characterization of potential glycosylation sites of the type 1 recombinant human immunodeficiency virus envelope glycoprotein (gp120) expressed in Chinese hamster ovary cells. J. Biol. Chem. 265, 10373-10382 (1990).
34. Iijima, S. & Ichihashi, T. Structural Instability of Ultrafine Particles of Metals. Phys. Rev. Lett. 56, 616-619 (1986).
35. Ajayan, P. M. & Marks, L. D. Quasimelting and Phases of Small Particles. Phys. Rev. Lett. 60, 585-587 (1988).
36. Doraiswamy, N. & Marks, L. D. Electron beam induced small particle transformations: temperature. Surf Sci. 348, 67-69 (1996).
37. Yacaman, M. J., Ascencio, J. A., Liu, H. B. & Gardea-Torresdey, J. Structure shape and stability of nanometric sized particles. J. Vac. Sci. Technol. B 19, 1091-1103 (2001).
38. Kaltenbach, J. P., Kaltenbach, M. H. & Lyons, W. B. Nigrosin as a dye for differentiating live and dead ascites cells. Exp. Cell Res. 15, 112-117 (1958).
39. Kwong, P. D. et al. Structure of an HIV gp120 envelope glycoprotein in complex with the CD4 receptor and a neutralizing human antibody. Nature 393, 648659 (1998).

What is claimed is:

1. An anti-viral composition comprising one or more noble metal nanoparticles-conjugated to a protein.

2. The composition of claim 1, wherein the nanoparticles are provided at a concentration of at least about 31 g/mL or greater.

3. The composition of claim 1, wherein the nanoparticles are made available in a solution, suspension, cream, ointment, lotion, enema, elixir, syrup, emulsion, gum, insert, suppository, jelly, foam, paste, pastille, spray, magma or poultice.

4. The composition of claim 1, wherein the nanoparticles are packaged for immediate release, extended release and combinations thereof.

5. The composition of claim 1, wherein the noble metal comprises gold, silver, platinum alloys and combinations thereof.

6. The composition of claim 1, wherein the nanoparticles are enveloped in a single dose.

7. The composition of claim 1, wherein the nanoparticles are disposed in or about a condom.

8. The composition of claim 1, wherein the nanoparticles are packed into a capsule, caplet, softgel, gelcap, suppository, film, granule, gum, insert, pastille, pellet, troche, lozenge, disk, poultice or wafer.

9. The composition of claim 1, wherein over 80% of the nanoparticles are released within about 60 minutes.

10. The composition of claim 1, wherein the nanoparticles are provided for immediate release which comprises release of over 90% of the nanoparticles within about 90 minutes.

11. The composition of claim 1, wherein nanoparticles are packaged for extended release comprising release of over 80% of the nanoparticles within about 60 minutes to about 8 hours.

12. A method for preventing anti-viral infections comprising the steps of:
resuspending in a pharmaceutically acceptable carrier one or more globular protein-noble metal nanoparticles to form an anti-viral composition; and
providing the anti-viral composition to a mammal.

13. A method of treating a patient suspected of having a viral infection comprising the steps of:
providing a patient suspected of being in need of anti-viral therapy with a composition comprising silver nanoparticles in a pharmaceutical acceptable carrier.

14. The method of claim 13, wherein the noble metal comprises silver, gold or platinum.

15. A method of making a protein-noble metal nanoparticles comprising the steps of:
mixing a noble metal nitrate and a halo-borohydride in the presence of a lower aliphatic alkyl alcohol.

16. The method of claim 15, wherein the protein and noble metal particle are at a ration of between about 1:28 to 28:1.

17. The method of claim 15, wherein the noble metal nitrate comprises AgNO₃.

18. The method of claim 15, wherein the noble metal comprises silver, gold, platinum, mixtures, alloys and combinations thereof.

19. The method of claim 15, wherein the protein comprises a globular protein.

20. The method of claim 15, wherein the protein is selected from albumin, immunoglobulin, a clotting factor, hemoglobin, mixtures and combinations thereof.

21. An anti-viral composition comprising one or more noble nanoparticles-conjugated to a globular protein.

22. The composition of claim 21, wherein the nanoparticles are provided at a concentration of at least about 3 μg/mL or greater.

23. The composition of claim 21, wherein the nanoparticles are made available in a solution, suspension, cream, ointment, lotion, enema, elixir, syrup, emulsion, gum, insert, suppository, jelly, foam, paste, pastille, spray, magma or poultice.

24. The composition of claim 21, wherein the nanoparticles are packaged for immediate release.

25. The composition of claim 21, wherein the nanoparticles are packaged for extended release.

26. The composition of claim 21, wherein the nanoparticles comprise 1-2 nm gold nanoparticles.

27. The composition of claim 21, wherein the nanoparticles comprise 2-5 nm silver nanoparticles
